(19) United States
(12) Patent Application Publication
(10) Pub. No.: US 2022/0390435 A1
(43) Pub. Date: Dec. 8, 2022

(54) COMPOSITIONS FOR PATIENT SPECIFIC IMMUNOTHERAPY

(71) Applicant: VITROSCAN B.V., Leiden (NL)

(72) Inventors: Abbas JARIANI, Leiden (NL); Fanny GRILLET, Leiden (NL); Willemijn VADER, Leiden (NL)

(21) Appl. No.: 17/771,254

(22) PCT Filed: Oct. 23, 2020
(86) PCT No.: PCT/NL2020/050661

§ 371 Date: Apr. 22, 2022

(30) Foreign Application Priority Data
Oct. 26, 2019 (NL) 2024107

(51) Int. Cl. G01N 33/50; C07K 16/28; C12N 5/09; A61K 39/395; A61K 45/06

(52) U.S. Cl. CPC G01N 33/5044; C07K 16/2818; C12N 5/0693; G01N 33/502; A61K 39/39558; A61K 45/06; C07K 2317/21; C07K 2317/76; C12N 2513/00; A61K 2039/55

(57) ABSTRACT

The present invention provides a pharmaceutical composition for the treatment of patients having ovarian cancer, lung cancer or mesothelioma and showing a Selection Factor of −30% or below, comprising a therapeutically effective amount of ipilimumab, and optionally a pharmaceutically acceptable diluent or carrier, wherein the patient is selected on the basis of a positive response to an ex vivo three-dimensional (3D) patient derived tumour culture, the method comprising: (a) preparing a three-dimensional, optionally size-normalised, tumour culture from a patient-derived tumour sample in a multitude of replicates; (b) adding one or more immunotherapeutic agents to the culture, and (c) culturing for a pre-defined time period; and (d) determining the effect that the one or more immunotherapeutic agents has on the tumour cell aggregates by measuring the total area of objects in the culture that are above a threshold area associated with tumour cell aggregates, and the total area of objects that are below a threshold associated with immune cells, using three-dimensional imaging of the cell culture; wherein if following culturing with a composition comprising ipilimumab, the total area of the large objects decreases and/or the total area of the small objects increases relative to a control the patient is treated with ipilimumab.

### TECHNICAL FIELD

The present invention relates generally to the field of targeted immunotherapy, and more specifically to compositions comprising antibodies to counteract or induce immune tolerance of cancer cells for patients that have shown an increased responsiveness.

It further relates to a method for measuring patient specific responses to immunomodulation in in vitro tumour cell cultures. It further relates to a method for predictive testing, and to immunotherapy or chemo-immunotherapy for a patient group selected by the method.

### BACKGROUND OF THE INVENTION

Immunotherapy is a therapy based on the administration of agents that trigger or enhance immune responses to tumour cells. However, indiscriminate triggering of an immune response has been found to have many adverse effects, and in spite of some successes, response rates for many cancer indications are relatively low. It therefore appears that immunotherapy is not widely available for all patients.

Immunomodulation on a cellular basis is a mechanism that protects cells or an organism by limiting and modulating the immune reaction, which is directed at eliminating foreign pathogens while maintaining self-tolerance. In this process, immune checkpoints playing a crucial role in immunomodulation. Immune checkpoints are a group of extracellular membrane-bound proteins expressed on immune effector cells, either inhibiting or stimulating effector cell proliferation. The checkpoints are involved in eliminating foreign pathogens while maintaining self-tolerance. Therapeutic antibodies designed to block or activate immune checkpoints have allowed a new approach for the treatment of cancer and other diseases.

However, while checkpoint blockade immunotherapies were found very successful for some cancer patients, a large number of patients, do not benefit from these relatively costly therapies, but on the contrary suffer from severe adverse events.

Accordingly, there remains a need for predictive tests for anti-proliferation drugs, more specifically for immunotherapeutic agents, and/or synergistic combinations of anti-checkpoint inhibitors for the treatment of patients with proliferative diseases.

It is hence an object of the invention to provide efficacious immunotherapeutic treatment regimens wherein one or more immunotherapeutic agent(s) for the treatment of proliferative diseases, are administered to patients that react positively to this treatment.

Accordingly, the present inventors made an effort to solve the problems of the related art as described above. As a result, the present inventors confirmed that in the case where a patient-derived cancer cell culture is subjected to a screening method including three-dimensional culture, patient-specific immunotherapeutic agents are capable of being efficiently selected by using a sample of patient derived cancer cells as compared to the existing screening method.

### SUMMARY OF THE INVENTION

In a first aspect, the present invention provides compositions comprising ipilimumab for use in the treatment of a patient that has shown a selective response to ipilimumab in the present specimen in-vitro test method.

In yet a further aspect, the present invention relates to compositions comprising ipilimumab for use in the treatment of a patient that has shown a selective response to ipilimumab in the in vitro test method.

Accordingly, in a first aspect, the present invention relates to a method for measuring the immune-mediated effect of one or more immunotherapeutic agents on patient derived tumour cultures, the method comprising: (a) preparing a three-dimensional size-normalised culture in a multitude of replicates comprising patient derived tumour cell aggregates in a growth medium; (b) adding the one or more immunotherapeutic agents to the culture, and (c) culturing for a pre-defined time period; and (d) determining the effect that the one or more immunotherapeutic agents has on the tumour cell aggregates by measuring the total area of objects in the culture that are above about 420 μm², and the total area of objects that are below about 160 μm², using three-dimensional imaging of the cell culture, and (e) identifying the patients that are responsive to one or more immunotherapeutic agents.
In a second aspect, the present invention also relates to a 3-dimensional cell culture obtainable according to the above method.
In a further aspect, the present invention also relates to a kit comprising the cell culture according to the invention and an imaging analysing apparatus.

In a third aspect, the present invention relates to an in-vitro ovarian and mesothelioma tumour cell culture, comprising three-dimensional cell aggregates.

In a further aspect, the present invention relates to an in-vitro lung tumour cell culture, comprising three-dimensional cell aggregates.

In a further aspect, the present invention relates to an in-vitro ovarian, lung and mesothelioma tumour cell culture, comprising three-dimensional cell aggregates.

In a further aspect, the present invention relates to a test method for testing patient specific drug efficacy.

In yet a further aspect, the present invention relates to ipilimumab for use in the treatment of a patient that has shown a selective response to ipilimumab in the in vitro test method.

In yet a further aspect, the present invention relates to combinations comprising ipilimumab for use in the treatment of a patient that has shown a selective response to ipilimumab in the in vitro test method.

In another aspect, the present invention relates to a method for identifying agents having patient specific anticancer activity against ovarian cancer and mesothelioma cells comprising selecting at least one test agent, contacting a plurality of ex vivo patient derived ovarian cancer or mesothelioma cell aggregates with the one or more test agents, determining the number, size and viability of tumour cell aggregates in the presence and absence of the test agent, and identifying an agent having anticancer activity if the number, size and viability of aggregates with a size above 420 μm² is lower than in the presence of the agent than in the absence of the agent.

In another aspect, the present invention relates to a method for identifying agents having patient specific anticancer activity against ovarian cancer, lung cancer and mesothelioma cells comprising selecting at least one test agent, contacting a plurality of ex vivo patient derived ovarian cancer or mesothelioma cell aggregates with the one or more test agents, determining the number, size and viability of tumour cell aggregates in the presence and absence of the test agent, and identifying an agent having anticancer activity if the number, size and viability of aggregates with a size above 420 μm² is lower than in the presence of the agent than in the absence of the agent.

In another aspect, the present invention relates to a method for identifying agents having patient specific anticancer activity against lung cancer comprising selecting at least one test agent, contacting a plurality of ex vivo patient derived lung cancer aggregates with the one or more test agents, determining the number, size and viability of tumour cell aggregates in the presence and absence of the test agent, and identifying an agent having anticancer activity if the number, size and viability of aggregates with a size above 420 μm² is lower than in the presence of the agent than in the absence of the agent.

In some aspects, the invention provides methods for treating a patient or subject with an immune checkpoint modulator wherein the subject is identified to have a tumour, wherein the presence of the patient's immune cells may permit application of successful therapy with an immune checkpoint modulator, comprising a step of selecting for receipt of the therapy a subject identified as having a tumour and immune cells susceptible to treatment with administered immune checkpoint modulators, wherein an improvement comprises administering therapy to a subject identified as having a cancer. In some embodiments, the invention provides methods for treating a cancer selected from the group consisting of carcinoma, sarcoma, myeloma, leukaemia, or lymphoma, the methods comprising a step of administering immune checkpoint modulator therapy to a subject identified as having a cancer and immune system susceptible to treatment with administered immune checkpoint modulators.

### SHORT DESCRIPTION OF THE DRAWINGS

The following figures are presented for the purpose of illustration only, and are not intended to be limiting:

FIG. 1 shows samples where Ipilimumab appears to be effective in activating native immune cells to kill ovarian cancer tumoroids. Plots in the top row indicate total area from objects larger than 422 μm². This metric indicates the abundance and survival of tumour cell clusters. The plots on the bottom row indicate the total area of objects smaller than 160 μm². This metric indicates abundance of immune cells. The indicated p-values correspond to one-sided Wilcoxon test.

FIG. 2—Similar to FIG. 1, but for the ovarian cancer samples where Ipilimumab does not show effectiveness in activating native immune cells to kill tumoroids.

FIG. 3—Similar to FIG. 1, but for a Mesothelioma tumour sample showing immune-mediated tumoroid killing effect when treated with Ipilimumab.

FIG. 4—Similar to FIG. 1, but for an ovarian tumour sample showing immune-mediated tumoroid killing effect when treated with ADU-S100, and pembrolizumab, and combinations thereof.

FIG. 5—Similar to FIG. 1 and same sample as FIG. 4, but for an ovarian tumour sample showing immune-mediated tumoroid killing effect when treated with ADU-5100, and pembrolizumab, and combinations thereof.

FIG. 6—Similar to FIG. 1, but for a lung tumour sample showing immune-mediated tumoroid killing effect when treated with ADU-5100, pembrolizumab, ipilimumab, and nivolumab, and combinations thereof.

FIG. 7—IFN-γ production is increased in conditions treated with Staphylococcal enterotoxin A (SEA, (positive control) and Ipilimumab. IFN-γ is secreted by activated T-cells.

FIG. 8—Tumoroids' area decrease (%) as Selection Factor. This metric is conditionally dependent on statistical increase in number (total area) of small objects due to Ipilimumab treatment, and decrease in area of the tumoroids (large objects) as detailed in Formula I. The filled circle symbols represent the metric for Ipilimumab treatment, and the plus symbols represent the metric for SEA treatment (positive control). In some experiments, Ipilimumab was tested in combination with Nivolumab or Pembrolizumab. In the shown experiments Nivolumab and Pembrolizumab do not show any effect when used alone, but in this figure, we show their combination with Ipilimumab as a biological replicate for Ipilimumab effect, in order to indicate the robustness of this metric.

FIG. 9—Tumoroid's area decrease (%) as Selection Factor. This metric is conditionally dependent on statistical increase in number (total area) of small objects due to treatment, and decrease in area of the tumoroids (large objects) as detailed in Formula I. Each row is a distinct treatment (monotherapy or combination) and each column indicates a sample. All samples are ovarian ascites unless indicated otherwise; suffix “_T” indicates a solid tumour sample and suffixes “_I” and _II” indicate the first and second sample from the same patient, respectively. The shade of grey and number in each cell indicate the selection factor value. Hatched cells indicate that treatment was not tested on the sample.

### DETAILED DESCRIPTION OF THE INVENTION

It must also be noted that as used herein and in the appended claims, the singular forms “a”, “an”, and “the” include plural reference unless the context clearly dictates otherwise. Thus, for example, reference to a “cell” is a reference to one or more cell and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

“Optional” or “optionally” means that the subsequently described event or circumstance may or may not occur, and that the description includes instances where the event occurs and instances where it does not.

“Administering” when used in conjunction with a therapeutic means to administer a therapeutic to a patient whereby the therapeutic positively impacts the tissue to which it is targeted. Thus, as used herein, the term “administering”, when used in conjunction with cancer therapy, can include, but is not limited to, providing a treatment into or onto the target tissue; providing a treatment systemically to a patient by, e.g., intravenous injection whereby the therapeutic reaches the target tissue; providing a treatment in the form of silencing expression of a specific gene. “Administering” a composition may be accomplished orally, by injection, topical administration, or by these methods in combination with other known techniques.

The term “tissue” refers to any aggregation of similarly specialized cells which are united in the performance of a particular function.

As used herein the term “modulator” means any active agent that modulates the activation state of immune cells, thereby modulating an immune response such as cell killing by effector cells, such as cytotoxic T-cells) in a subject (e.g., a human subject).

As used herein, the term “therapeutic” means an agent utilized to treat, combat, ameliorate, prevent or improve an unwanted condition or disease of a patient. Embodiments of the present invention are directed to regulating cancer cell growth.

The methods and devices of the present invention do not require staining of cells with toxic dyes, and therefore, allows for observation of cell growth or inhibition of cell growth in real time.

The one or more immunotherapeutic agents may be added to the culture in combination with other known anti-cancer therapies or compounds. Preferably, the anti-cancer compound is olaparib.

The term “Ipilimumab” relates to ipilimumab (Yervoy), a monoclonal antibody that targets the protein receptor cytotoxic T-lymphocyte-associated protein 4 (CTLA-4).

A preferred ipilimumab is the human monoclonal antibody 10D1 (also referred to as MDX-010 and ipilimumab and available from Medarex, Inc., Bloomsbury, N.J.) is disclosed for instance in WO 01/14424.

As noted elsewhere herein, the administration of Ipilimumab and one or more other active anti-CTLA4 antagonists may be administered either alone or in combination with known anti-cancer therapies. Advantageously, this one or more immunotherapeutic agents according to the invention comprises Ipilimumab as a monotherapy or in combination with other immunomodulators. Preferably, the one or more other immunotherapeutic agents is selected from the group consisting of: pembrolizumab and nivolumab. Preferably, the one or more other immunotherapeutic agents is selected from the group consisting of: pembrolizumab, ADU-S100 and nivolumab. Preferably, the one or more other immunotherapeutic agents is selected from the group consisting of durvalumab, atezolizumab, tremelimumab, spartalizumab, cemiplimab, pembrolizumab, ADU-S100 and/or nivolumab. Preferably, the one or more other immunotherapeutic agents is ADU-S100.

Preferably, the known anti-cancer therapy is the use of olaparib for treatment in cancer. The term “olaparib” relates to olaparib (Lynparza, available from AstraZeneca, Cambridge, UK), a poly ADP ribose polymerase (PARP) inhibitor.

An individual that shows a significant response to an immunotherapeutic treatment is a patient who is affected with a cancer and who will show a clinically significant response after receiving said anticancer treatment; the clinically significant response may be assessed by clinical examination (body weight, general status, pain and palpable mass, if any), biomarkers and imaging studies (ultrasonography, CT scan, PET scan, MRI). According to a specific embodiment, the individual is a patient with ovarian cancer or mesothelioma.

“Selection Factor” herein means the effect that the agent shows on the number and size of larger cell aggregates, i.e. cell aggregates larger than 420 μm² in the cell culture; conditioned on statistically significant decrease in abundance of these larger objects and as well conditioned on statistically significant increase in abundance of objects smaller than 160 μm². Without wishing to be bound to any particular theory, it is believed that the increased abundance of smaller objects corresponds at least in part to activation of immune cells. For this purpose, the sum of area in all the tumoroids with an area larger than 420 μm² is calculated in each of the multitude of samples, such as preferably in each well of a standard 384-well plate. If this sum of areas is not statistically significantly lower across the replicates in immunomodulator treatment compared to the negative control, then the value of Selection Factor is assigned as zero. The statistical test used here is applied as a one-sided Wilcoxon test, which does not assume normality of the data. A Wilcoxon signed-rank test is a non-parametric statistical hypothesis test used to compare two related samples, matched samples, or repeated measurements on a single sample to assess whether their population mean ranks differ i.e. it is a paired difference test.

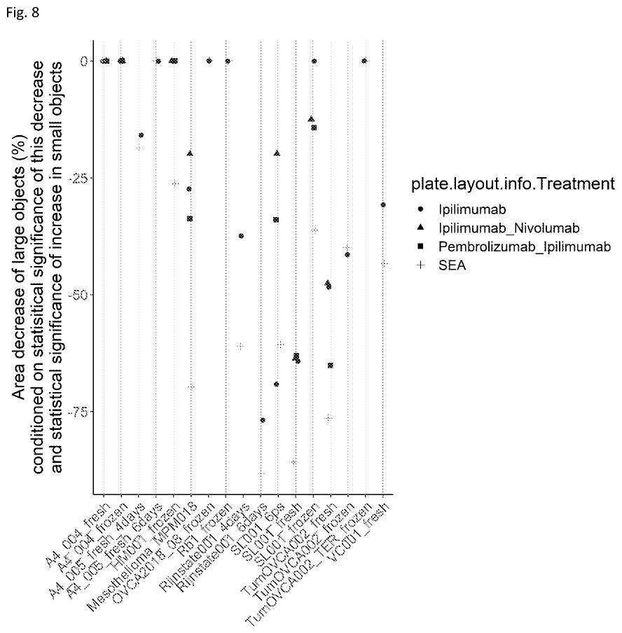

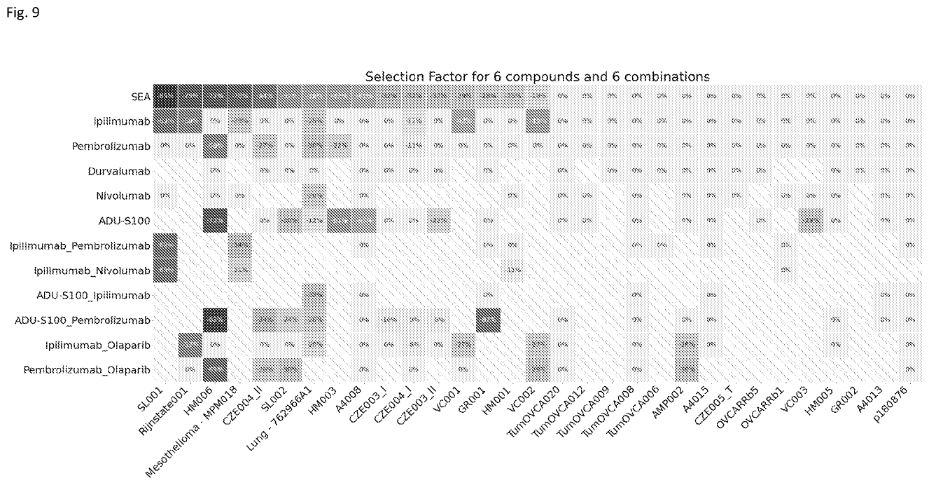

In the next step, it is tested whether there is statistically significant increase in number of immune cells (area<160 μm²) in response to the checkpoint inhibitor, e.g. Ipilimumab. For this purpose, the sum of the area of the small objects with an area<160 μm² in each well of the 384-well plate is calculated. If this sum of areas is not statistically significantly higher across the replicates in Ipilimumab treatment compared to the negative control, then the Selection Factor is assigned as zero. In case both these two statistical test checks are met, we assign Selection Factor as percentage of decrease in the across-replicate median of decrease in area of tumoroids (large objects). The measured metric across different samples are shown in FIGS. 8 and 9 for ipilimumab, pembrolizumab, nivolumab, ADU-S100, and olaparib, which showed that these compounds were, alone or in combination, able to start an immune reaction that effectively reduced the number of tumour cells, based only on the immune system of a naïve patient sample.

As used herein, the term “cancer” refers to or describe the physiological condition in mammals in which a population of cells are characterized by unregulated cell growth. Examples of cancer include, but are not limited to, carcinoma, lymphoma, blastoma, sarcoma, and leukaemia. More particular examples of such cancers include metastatic or non-metastatic disease of any types of lung cancer, peritoneal cancer, gastrointestinal cancer, pancreatic cancer, melanoma, glioblastoma, cervical cancer, ovarian cancer, mesothelioma, liver cancer, bladder cancer, liver cancer, breast cancer, colon cancer, colorectal cancer, endometrial or uterine carcinoma, salivary gland carcinoma, kidney cancer, liver cancer, prostate cancer, vulvar cancer, thyroid cancer, hepatic carcinoma and head and neck cancer.

The term “treatment” herein refers to any reduction of the progression, severity and/or duration of cancer.

The present invention also encompasses a pharmaceutical composition useful in the treatment of proliferative diseases, more specifically cancer, more specifically ovarian cancer, lung cancer or mesothelioma, yet more specifically ovarian cancer or mesothelioma, even more specifically lung cancer, comprising the administration of a therapeutically effective amount of the agents, or agent combinations according to the invention, with or without pharmaceutically acceptable carriers or diluents. The pharmaceutical compositions according to the invention advantageously comprise an anti-proliferative agent or agents, and a pharmaceutically acceptable carrier.

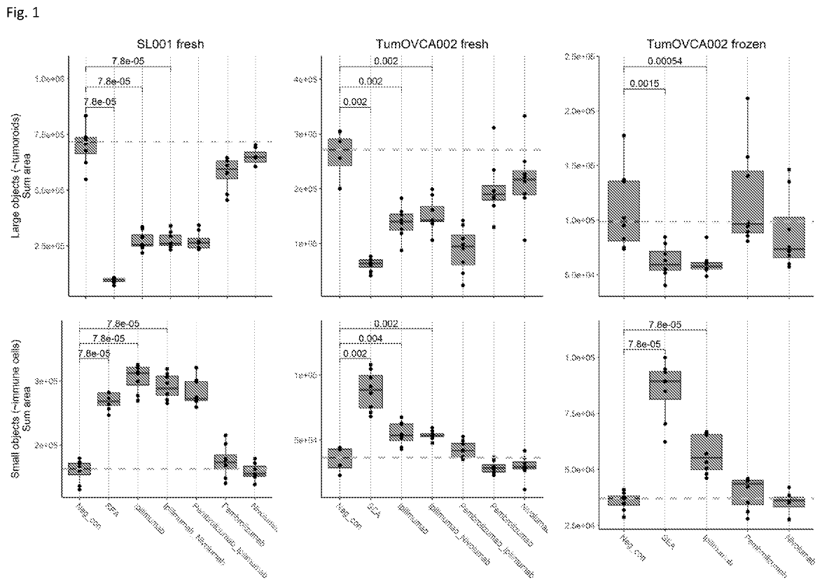

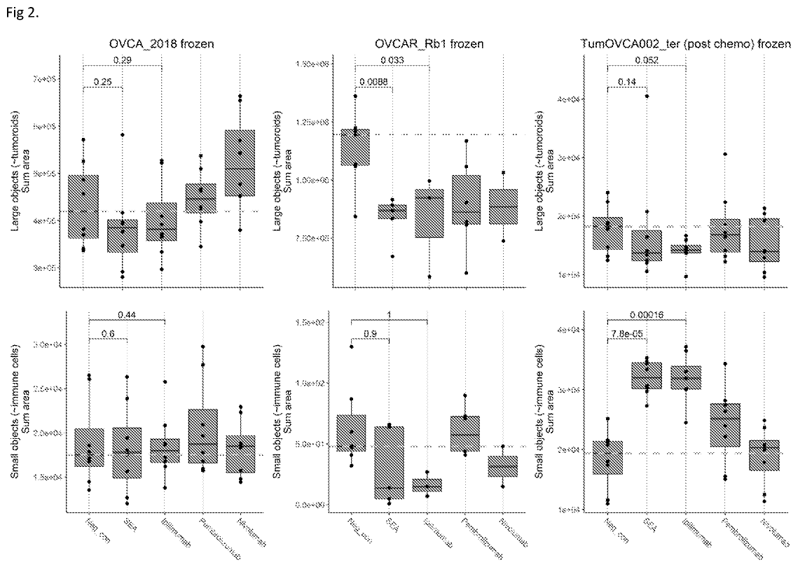

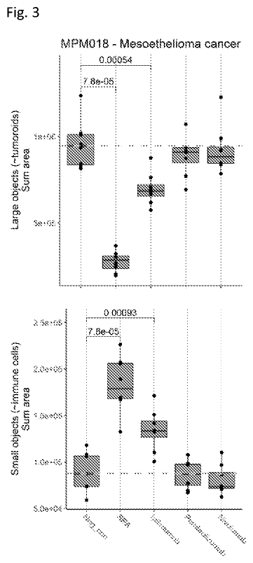

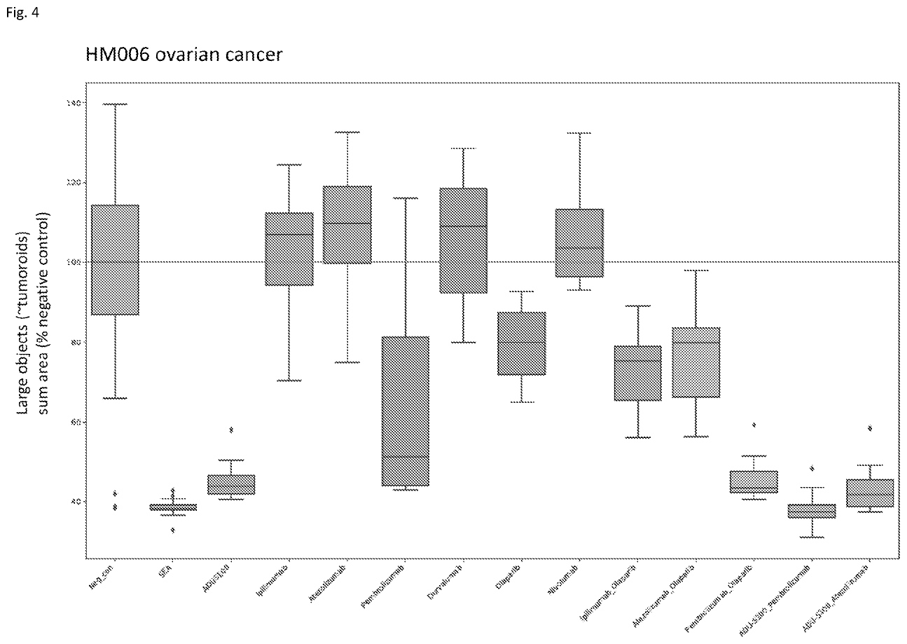

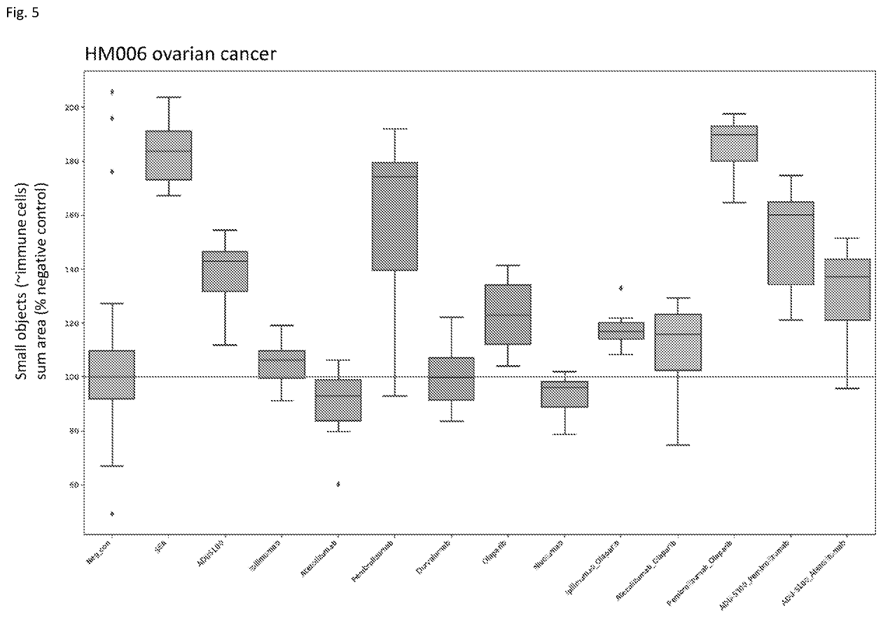

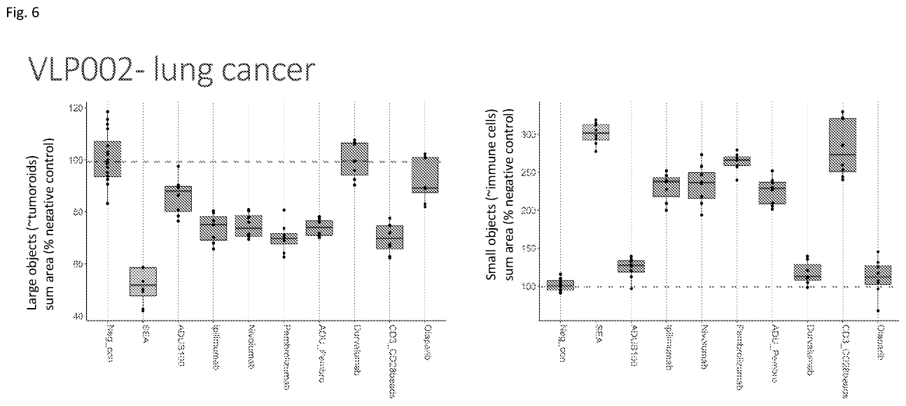

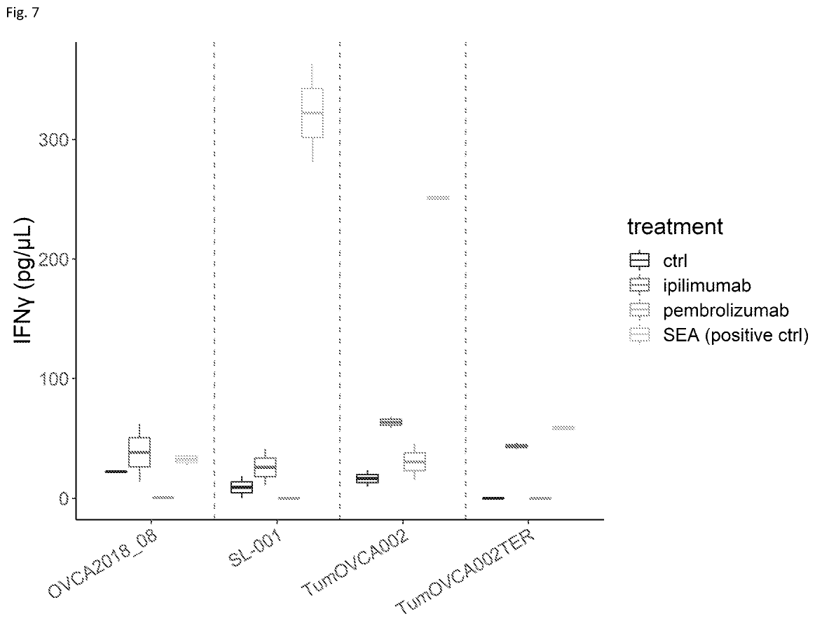

Given the high cost, significant side effects, and patient-to-patient response variability of immunotherapeutic agents for cancer treatment, it is highly appealing to develop an in-vitro assay to predict response of individual patients to these treatments. Applicants have developed an assay which can gauge immunotherapeutic agents for treatment efficiency for a.o. ovarian, lung and mesothelioma cancer patients. This assay is based on ex vivo 3D culturing of individual patient's tumour tissues, i.e. derived from fresh or cryopreserved samples, in combination with 3D imaging, and subsequent morphological characterization of cell types in the sample as set out for instance in Booij, T. H., Bange, H., Leonhard, W. N., Yan, K., Fokkelman, M., Kunnen, S. J., . . . Price, L. S. (2017), High-Throughput Phenotypic Screening of Kinase Inhibitors to Identify Drug Targets for Polycystic Kidney Disease, SLAS DISCOVERY: Advancing Life Sciences R&D, 22(8), 974-984. Representative results from this assay are shown in FIGS. 1-6. For the samples in FIG. 1, Ipilimumab showed effectiveness in activating immune cells for ovarian cancer, in order to kill the tumoroids, while similar effect were not observed in samples shown in FIG. 2. FIG. 3 shows a sample where we see killing effect in a mesothelioma cancer sample. FIG. 4 shows a sample where we see a killing effect, i.e. area size reduction of tumoroid clusters, in an ovarian tumour sample when treated with ADU-S100, and pembrolizumab, and combinations thereof. FIG. 5 shows the same sample of FIG. 4, where we see area size increase of small objects in an ovarian tumour sample when treated with ADU-S100, and pembrolizumab, and combinations thereof. FIG. 6 shows a sample where we see killing effect in a lung cancer tumour sample when treated with ADU-S100, and pembrolizumab, ipilimumab, nivolumab, and combinations thereof. FIG. 7 shows that the observed tumour killing effect is accompanied by IFN-γ production, an evidence for presence of activated T-cells. Based on these observations, applicants found that statistical test-conditioned decrease in tumoroid area of tumoroid clusters larger than approximately 420 μm² was suitable as Selection Factor as a read-out for Ipilimumab efficacy in each patient, thereby effectively allowing for a patient selection.

In the present method, preferably, a multitude of replicates are prepared and analysed in parallel.

Advantageously, the multitude of samples are prepared from the tissue or fluid sample in parallel, wherein each sample is placed in a well on a microtiter plate, and wherein each sample comprises of from 100 to 300 aggregates in a volume of from 1 to 20 μl of a suitable growth matrix, preferably a hydrogel and of from 19 to 40 μl of a suitable medium, wherein the total volume of a sample is 60 μl.

Advantageously, the multitude of samples are prepared from the tissue or fluid sample in parallel, wherein each sample is placed in a well on a microtiter plate, and wherein each sample comprises of from 1 to 800 aggregates, preferably of from 10 to 800 or of from 1 to 500, more preferably of from 10 to 100 or of from 100 to 500, even more preferably of from 200 to 300 aggregates, in a volume of from 1 to 20 μl of a suitable growth matrix, preferably a hydrogel and of from 19 to 40 μl of a suitable medium, wherein the total volume of a sample is 60 μl.

Preferably, step (a) comprises providing a test sample comprising patient-derived tumour cellular material and immune cells from a mammalian tumour tissue or fluid sample by: (i) subjecting the sample to mild shearing and/or filtration to obtain homogenized cellular material isolated cells and cell aggregates ranging from 30-100 μm in diameter, which may be variable depending on the starting material; and (ii) enriching of the sample is achieved by filtration to reduce the number of aggregates larger than 100 μm in diameter, and/or reduce aggregates smaller than 30 μm, advantageously by passing the sample through a mesh filter with the appropriate mesh size; (iii) embedding the homogenized cellular material with a growth medium for a period and under conditions suitable for three-dimensional cell culture comprising aggregates of a surface area of more than 420 μm²;

Preferably, the tissue sample may be directly employed after sampling and optional transport, or as a sample cryopreserved according to a standard protocol for preserving viability of immune cells present in the sample, or wherein the sample is split into a fresh sample and a cryopreserved sample for correlation of the data at a later point in time. Advantageously, wherein normalizing the average size of the sample contents by subjecting the sample to mild shear and/or filtration to enrich for the preferred size of aggregates ranging from 30-100 μm in diameter in the growth medium. Preferably the shearing is conducted by passing the tumour sample through a restrictive orifice, such as a syringe needle one or more times, such as at least 3 times through a G25 syringe. The shearing may also be conducted by passing the tumour sample through other types of syringes or through a pipette one or more times, such as at least 3 times. Filtration is performed to by using filters with appropriate mesh size.

Preferably, the samples comprising tumour cells are derived from a from a patient with metastatic or non-metastatic cancer. More particular examples of such cancers include any types of lung cancer, peritoneal cancer, gastrointestinal cancer, pancreatic cancer, melanoma, glioblastoma, cervical cancer, ovarian cancer, mesothelioma, liver cancer, bladder cancer, breast cancer, colon cancer, colorectal cancer, endometrial or uterine carcinoma, salivary gland carcinoma, kidney cancer, prostate cancer, vulvar cancer, thyroid cancer, hepatic carcinoma and head and neck cancer, preferably cancer is selected from ovarian cancer, lung cancer and mesothelioma, preferably cancer is selected from ovarian cancer and mesothelioma, preferably cancer is lung cancer. Preferably, the tumour cell culture comprises a naïve sample is derived from resected tumour specimen, tumour biopsies or malignant fluids (e.g. ascites, pleural effusion). Preferably, the three-dimensional culture comprises tumour cells and immune cells. Preferably, the patient derived tumour sample is the only source of immune cells in an ex vivo three-dimensional (3D) patient derived tumour culture. Ex vivo 3D patient derived tumour cultures in the art are commonly analysed on immune-mediated effects of one or more immunotherapeutic agents by adding an additional exogenous source of immune cells and therefore they do not solely rely on the immune cells present in the ex vivo 3D patient derived tumour culture. The ex vivo 3D patient derived tumour cultures preferably do rely only on the native immune cells that are present in the patient derived tumour sample after isolation from a patient.

Preferably, the three-dimensional culture comprising ex vivo tumour aggregates in a hydrogel is prepared by subjecting a tumour sample to shearing and/or filtration, to yield a cell culture comprising cells and cell aggregates ranging from 30-100 μm in diameter.

Preferably, the three-dimensional culture comprising ex vivo tumour aggregates in a hydrogel is prepared by subjecting a tumour sample to shearing and/or filtration, to yield a cell culture comprising cells and cell aggregates substantially ranging from 30-100 μm in diameter. A person skilled in the art knows that fully homogenising a tumour sample by shearing and/or filtration is difficult and a standard normal distribution is more likely to be achieved instead, hence the preference to yield a cell culture comprising cells and cell aggregates substantially ranging from 30-100 μm in diameter.

Preferably, the culturing period in step (c) is between about 3 and 7 days. Preferably, the objects that have a surface area of above 420 μm² are tumour cell aggregates or tumoroids and the object that are below about 160 μm² are considered immune cells.

Preferably, prior to the 3D imaging, the cell culture is stained, such as with actin staining reagents like tetramethyl rhodamine(TRITC)-phalloidin. Other staining reagents can be actin staining reagents like rhodamine-phalloidin or deoxyribonucleic acid (DNA) or cell nucleus staining reagents like 4′,6-diamidino-2-phenylindole (DAPI) or preferably Hoechst dyes such as Hoechst 33258.

Preferably, step (d) further comprises assessing the viability and/or size of the tumour cell aggregates of a surface area of more than 420 μm² in the presence or absence of the immunotherapeutic agents and/or anti-proliferation agent tested to create comparative data on viability and/or size of the tumour cell aggregates in presence or in absence of the immunotherapeutic and/or anti-proliferation agent, and relating the data obtained to values indicative of immunotherapeutic and/or anti-proliferation agent activity for reducing/increasing viability and/or size of the tumour cell aggregates.

Preferably, step (e) comprises optical scanning of the cultured sample with an automated computer-controlled multifocal fluorescence microscope.

The method according to the invention further preferably comprises providing the sample in a vessel aligned with and functionally coupled to the automated computer-controlled multifocal microscope; determining volumetric imaging parameters; directing excitation light onto a region of interest in the sample; scanning the fluorescence response light across a first portion of the sample; imaging a plurality of layers of the sample in a first volume of the sample in the region of interest to provide first image data; sectioning the first portion of the sample; scanning the excitation light across a second portion of the sample; imaging a second plurality of layers of the sample in a second volume of the sample to provide second image data; and processing the first image data and the second image data to form a three-dimensional image of the sample.

Advantageously, step (d) comprises measuring the effect of the one or more immunotherapeutic agents on viability and/or size of tumour cell aggregates, the method comprising: i) staining of the cell culture with a fluorescence marker and measuring the fluorescence intensity to determine the total area of stained objects in the culture that are above 420 μm² and below 160 μm², and ii) capturing a layered fluorescent image of the stained sample; iii) and measuring the object intensity of the luminescent surface areas in the sample; and iv) determining the luminescent surface areas. Advantageously, two fluorescence markers are used, preferably one marking actin and the other marking the cell nucleus or DNA. Advantageously, the sum of area of all tumoroids with an area larger than 420 μm² in each sample is calculated, and wherein it is determined if the sum of all areas is statistically significantly lower across the replicates comprising the same components compared to the negative control. Advantageously, the tumour-reducing effect is derived by calculating the percentage decrease of tumoroid area as a median of a multitude of parallel tests within each replicate, and the median as calculated across the replicates, wherein the tumoroids are distinguished by an area threshold of 420 μm² according to formula I.

$$I ) Wilcoxon test : Does total area of large objects decrease ( p < 0.05 ) in treatment condition compared to the negative control ?$$

$$‐ No -> Selection Factor = 0$$

$$II ) Wilcoxon test : Does total area of small objects increase ( p < 0.05 ) in treatment condition compared to the negative control ?$$

$$‐ No -> Selection Factor = 0$$

$$If ( I ) \& ( II ) are met \to Selection Factor = 100 * \frac{\begin{matrix}median\_replicate ( {\sum }_{area > 422 μ m 2}^{Treatment} object area ) - \\ median\_replicate ( {\sum }_{area > 422 μ m 2}^{Negative ctl} object area )\end{matrix}}{median\_replicate ( {\sum }_{area > 422 μ m 2}^{Negative ctl} object area )}$$

Herein, a Selection Factor below −30% indicates an effective treatment, and a patient responsive to the treatment. In this formulation the value of Selection Factor is conditioned by statistical increase in tumoroid area reduction (large objects) and immune cell amplification (small objects).

Advantageously, step (d) further segmenting the 3-dimensional culture into layers, capturing images of each layer, and deconvoluting the luminescence images of the layers to showing individual cells and cell aggregates in the culture.

Preferably, a decrease in the total area of objects that are above about 420 μm² and an increase in the total area of objects that are less than about 160 μm² compared to a control indicates that the one or more immunotherapeutic agent(s) is(are) effective at reducing the number of tumour cells.

The present invention also encompasses a pharmaceutical composition useful in the treatment of proliferative diseases, more specifically cancer, more specifically ovarian cancer, lung cancer and mesothelioma, yet more specifically ovarian cancer or mesothelioma, comprising the administration of a therapeutically effective amount of the agents, or agent combinations according to the invention, with or without pharmaceutically acceptable carriers or diluents. The pharmaceutical compositions according to the invention advantageously comprise an anti-proliferative agent or agents, and a pharmaceutically acceptable carrier. Advantageously, ipilimumab is administered at a dose of about 0.3 mg/kg.

Preferably, the threshold associated with the tumour cell aggregates is above about 420 μm², and wherein the threshold associated with immune cells is 160 μm².

In an embodiment, the tissue sample may be directly employed after sampling and optional transport, or as a cryopreserved sample according to a standard protocol for preserving viability of human cells present in the sample, or wherein the sample is split into a fresh sample and a cryopreserved sample for correlation of the data at a later point in time.

According to a preferred embodiment, step (d) comprises measuring the effect of the one or more immunotherapeutic agents on ex vivo patient derived 3D tumour cultures, by

i) staining of the cell culture with a fluorescence marker and measuring the fluorescence intensity to determine the total area of stained objects in the culture that are above about 420 μm² and below 160 μm², and

ii) capturing a layered fluorescent image of the stained sample;

iii) and measuring the object intensity of the fluorescent surface areas in the sample; and iv) determining the fluorescent surface areas.

Advantageously, two fluorescence markers are used, preferably one marking actin and the other marking the cell nucleus or DNA. Advantageously, the sum of area of all tumour aggregates with an area of about 420 μm² in each sample is calculated, and wherein it is determined if the sum of all areas is statistically significantly lower across the replicates comprising the same components.

Advantageously, the sum of area of all immune cells with an area smaller than about 160 μm² in each sample is calculated, and wherein it is determined if the sum of all areas is statistically significantly higher across the replicates comprising the same components, compared to the negative control.

Advantageously, the effect on tumour aggregates is derived by calculating the percentage decrease of tumour aggregate area as a median of multitude of parallel tests within each replicate, and the median as calculated across the replicates, wherein the tumour aggregates are distinguished by an area threshold of 420 μm² and immune cells are distinguished by having their area smaller than 160 μm² according to formula I herein above wherein a Selection Factor below −30% indicates an effective treatment, and a patient responsive to the treatment.

Advantageously, step (d) further comprises segmenting the 3-dimensional culture into layers, capturing images of each layer, and deconvoluting the luminescence images of the layers to enhance the image contrast and create segmentation masks for individual cells and cell aggregates in the culture.

According to a preferred embodiment, the present invention also relates to a composition according to the invention for use in a method for the treatment of female patients with recurrent epithelial ovarian cancer and showing a Selection Factor of −30% or below. Preferably, the composition may further comprise a synergistic and therapeutically effective amount of nivolumab and/or pembrolizumab.

According to a preferred embodiment, the present invention also relates to the compositions for use in treatment of a patient with metastatic or non-metastatic cancer, preferably lung cancer, peritoneal cancer, gastrointestinal cancer, pancreatic cancer, melanoma, glioblastoma, cervical cancer, ovarian cancer, liver cancer, bladder cancer, breast cancer, colon cancer, colorectal cancer, endometrial or uterine carcinoma, salivary gland carcinoma, kidney cancer, prostate cancer, vulvar cancer, thyroid cancer, mesothelioma, hepatic carcinoma and head and neck cancer, more preferably ovarian cancer, lung cancer or mesothelioma, more preferably ovarian cancer or mesothelioma, wherein the effect on tumour aggregates is derived by calculating the percentage decrease of tumour aggregate area as a median of multitude of parallel tests within each replicate, and the median as calculated across the replicates, wherein the tumour aggregates are distinguished by an area threshold of 420 μm² and immune cells are distinguished by having their area smaller than 160 μm² according to formula I herein above, wherein a Selection Factor below −30% indicates an effective treatment, and a patient responsive to the treatment.

Advantageously the tumour cell culture comprises a naïve sample is derived from resected tumour specimen, tumour biopsies or malignant fluids, such as ascites or pleural effusion.

Advantageously, the treatment with the composition may further comprises a chemotherapeutic or immunotherapeutic molecule, a small molecule kinase inhibitor, a hormonal agent, a vaccine, ionizing radiation, ultraviolet radiation, cryoblation, thermal ablation, or radiofrequency ablation.

The compositions of this invention may be formulated with conventional carriers and excipients, which will be selected in accord with ordinary practice. Tablets may contain excipients, glidants, fillers, binders and the like. Aqueous formulations are prepared in sterile form, and when intended for delivery by other than oral administration generally will be isotonic. All formulations may optionally contain conventional excipients, such as ascorbic acid and other antioxidants, chelating agents such as EDTA, carbohydrates such as dextrin, hydroxyalkyl cellulose, hydroxyalkyl methylcellulose, stearic acid and the like. The pH of the formulations may range from about 3 to about 11, but is ordinarily about 7 to 10.

While it is possible for the active ingredients to be administered alone it may be preferable to present them as pharmaceutical formulations.

The formulations of the invention, both for veterinary and for human use, comprise at least one active ingredient, e.g. a compound of the present invention, together with one or more acceptable carriers and optionally other therapeutic ingredients. The carrier or carriers must be acceptable in the sense of being compatible with the other ingredients of the formulation, and physiologically innocuous to the recipient thereof.

Pharmaceutical formulations according to the present invention may comprise one or more active agents of the invention together with one or more pharmaceutically acceptable carriers or excipients and optionally other therapeutic agents. Pharmaceutical formulations containing the active ingredient may be in any form suitable for the intended method of administration.

The formulations may include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets, lozenges or tablets each containing a predetermined amount of the active ingredient(s); as a powder or granules; as a solution or a suspension in an aqueous or non-aqueous liquid; or as an oil-in-water liquid emulsion or a water-in-oil liquid emulsion. The active ingredient(s) may also be administered as a bolus, electuary or paste. Formulations for oral use may be also presented as hard gelatin capsules where the active ingredient is mixed with an inert solid diluent, for example calcium phosphate or kaolin, or as soft gelatine capsules wherein the active ingredient is mixed with water or an oil medium, such as peanut oil, liquid paraffin or olive oil. Formulations suitable for topical administration in the mouth include lozenges comprising the active ingredient in a flavoured basis, usually sucrose and acacia or tragacanth; pastilles comprising the active ingredient in an inert basis such as gelatine and glycerine, or sucrose and acacia; and mouthwashes comprising the active ingredient in a suitable liquid carrier.

A tablet is typically made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient(s).

For external tissue administration, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogues.

The oily phase of emulsions of this invention may be constituted from known ingredients in a known manner. While the phase may comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl may be employed. These may be used alone or in combination depending on the properties required. Alternatively, high melting point lipids such as white soft paraffin and/or liquid paraffin or other mineral oils are used.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more colouring agents, one or more flavouring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth herein, and flavouring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavouring and colouring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavouring agents. Syrups and elixirs may be formulated with sweetening agents, such as glycerol, sorbitol or sucrose. Such formulations may also contain a demulcent, a preservative, a flavouring or a colouring agent.

The pharmaceutical compositions of the invention may be in the form of a sterile injectable preparation for preferably intravenous administration, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight/weight). The pharmaceutical composition can be prepared to provide easily measurable amounts for administration. For example, an aqueous solution intended for intravenous infusion may contain from about 3 to 500 μg of the active ingredient per millilitre of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations for rectal administration may be presented as a suppository with a suitable base comprising for example cocoa butter or a salicylate.

Formulations suitable for intrapulmonary or nasal administration have a particle size for example in the range of 0.1 to 500 μm (including particle sizes in a range between 0.1 and 500 μm in increments such as 0.5 μm, 1 μm, 30 μm, 35 μm, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of infections as described herein.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostatic agents and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients provided by the present invention the formulations of this invention may include other agents conventional in the art having regard to the type of formulation in question, for example those suitable for oral administration may include flavouring agents.

The compositions of the present invention may further comprise one or more pharmaceutically acceptable additional ingredient(s) such as alum, stabilizers, antimicrobial agents, buffers, colouring agents, flavouring agents, adjuvants, and the like. Compounds and compositions of the present invention may be administered orally or parenterally including the intravenous, intramuscular, intraperitoneal, subcutaneous, rectal and topical routes of administration.

Dosages of the active ingredients in the pharmaceutical compositions can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions of the present disclosure employed, the route of administration, the time of administration, the duration of the treatment, other drugs, compounds and/or materials used in combination with the particular compositions employed, the age, sex, weight, condition, general health and prior medical history of the subject being treated, and like factors. A physician, veterinarian or other trained practitioner, can start doses of the pharmaceutical composition at levels lower than that required to achieve the desired therapeutic effect and gradually increase the dosage until the desired effect is achieved. In general, effective doses of the compositions of the present disclosure, for the prophylactic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of multiple doses of any of the compositions described herein. In some embodiments, the composition is administered orally the subject once, twice, 3 times, 4 times, 5 times, 6 times, 7 times, 8 times, 9 times, or at least 10 times.

Aspects of the present disclosure include methods and compositions for the treatment of cancer in a subject, more specifically, in a preferred embodiment ovarian cancer, or mesothelioma, in a more preferred embodiment ovarian cancer, lung cancer, or mesothelioma, in another embodiment lung cancer. In some embodiments, the subject has cancer or is at risk of developing cancer. Examples of cancers that may be treated according to the methods provided herein, include without limitation, carcinoma, glioma, mesothelioma, melanoma (e.g., metastatic melanoma), lymphoma, leukaemia, adenocarcinoma, breast cancer, ovarian cancer, mesothelioma, cervical cancer, glioblastoma, multiple myeloma, prostate cancer, Burkitt's lymphoma, head and neck cancer, colon cancer, colorectal cancer, non-small cell lung cancer, small cell lung cancer, cancer of the oesophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease, AIDS-associated primary effusion lymphoma, neuroectodermal tumours, or rhabdomyosarcoma. In some embodiments of the methods provided herein, the cancer is prostate cancer, bladder cancer, non-small cell lung cancer, urothelial carcinoma, melanoma, Merkel cell cancer, or renal cell carcinoma. In some embodiments, the cancer is melanoma, non-small cell lung cancer (NSCLC), Hodgkin's lymphoma, head and neck cancer, renal cell cancer, bladder cancer, or Merkel cell carcinoma.

In some embodiments, the cancer is ovarian epithelial cancer and the anticancer therapy involves administering ipilimumab, preferably alongside a PD-1 inhibitor (e.g., pembrolizumab, nivolumab).

Advantageously, the method of the invention allows to determine whether a patient with cancer is likely to benefit from a treatment with ipilimumab. Formulation of medicaments, and the use of pharmaceutically acceptable excipients are known and customary in the art and for instance described in Remington; The Science and Practice of Pharmacy, 21nd Edition 2005, University of Sciences in Philadelphia. Ways of administration are known and customary in the art are for instance described in Remington; The Science and Practice of Pharmacy, 21st Edition 2005, University of Sciences in Philadelphia.

For oral use, the active compounds and compositions of this invention may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added.

In addition, sweetening and/or flavouring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic. For preparing suppositories according to the invention, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Liquid preparations include solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas. Also included are solid preparations which are intended for conversion, shortly before use, to liquid preparations for either oral or parenteral administration. Such liquid forms include solutions, suspensions and emulsions.

The compounds or compositions described herein may also be delivered transdermally. The transdermal compositions can take the form of creams, lotions, aerosols and/or emulsions and can be included in a transdermal patch of the matrix or reservoir type as are conventional in the art for this purpose.

The compositions of the present invention may also be used in conjunction with other well-known therapies that are selected for their particular usefulness against the condition that is being treated. A medicament comprising the agent may preferably provided together with general anti-cancer therapy. Examples of said general anti-cancer therapy are radiation, chemotherapy, antibody-based therapy or small molecule-based treatments. Combined treatment leads to an approach of killing the minority cancer stem cell population as well as the bulk of the tumour.

The general anti-cancer therapy can be provided before, during, or after the provision of a medicament comprising the agent.

The following, non-limiting examples serve to illustrate the present invention.

### Examples

The following examples show tumour sensitivity vis-à-vis immunotherapeutic agent treatment in three-dimensional tumour aggregates from patients. The tests were performed by immunotherapeutic agent exposure on fresh and cryopreserved ovarian carcinoma, lung carcinoma and mesothelioma-derived primary tumour cells.

Imaging was accomplished using a position-controlled inverted fluorescence microscope stage to enable automated precise localization of each spot incrementally in conjunction with image capture using a CCD camera. Once images are captured, image analysis software defined the area of interest, subtract background, threshold the image to identify cell aggregates as objects, create a region around these objects, and measure their area. These objects can correspond to tumour cell aggregates or immune cells.

Per experiment group sum of object sizes were also determined. In one embodiment of the invention, these analyses can be carried out with existing computer programs, and in another preferred embodiment, software macros automate these steps.

To test treatment efficacy of immunotherapeutic agents, a panel of different immunotherapeutic and anti-proliferation agents were subjected to a three-dimensional (3-D) multi-parametric assay, whereby the ex vivo patient-derived cell aggregates were cultured in extra-cellular based hydrogels in 384-well plates, and in presence and absence of the tested agents.

The culture methods preserve the complex three-dimensional phenotype that facilitates measurement of changes in morphological phenotypes in a high content screen. The aggregates were grown in the presence of the agents at different concentrations. A known immune-activator compound Staphylococcal enterotoxin A (SEA) was included as positive controls, and untreated cell aggregates as negative controls.

Effects on the cells were captured by staining the samples and by collecting 3D image stacks.

Analysis of the data, retaining spatial information, to generate a set of more than 100 different measured features, including the number, shape, and size of objects, cells and nuclei; sub-populations; cell apoptosis and invasion, resulted in an assessment of the Selection Factor indicating the efficacy of any particular treatment.

Preparation of the Cell Cultures

Resected tumour specimen, tumour biopsies or malignant fluids (e.g. ascites, pleural effusion) comprising tumour and immune cells were homogenized to 30-100 μm maximal diameter by shearing with a 25G needle or a pipette and/or filtration (can be variable depending on the starting material).

This step comprised using either freshly isolated aggregates or cryopreserved and thawed samples prepared by following a standard protocol for preserving the viability of human cells.

Samples were then transferred to a 384 screening well plate as follows, by contacting an aliquot comprising of from 1 to 500, more preferably of from 10 to 800, and again more preferably of from 200 to 300 cell aggregates with an extra-cellular matrix protein based hydrogel. In more detail, the aggregates were contacted with a gel composition comprising 50-70% matrigel, 0.2 to 1.0 mg/ml collagen, 5-15% NaHCO₃ and 10% HEPES 1M, in amount of about 80% to about 20% of cell aliquot in its medium. The gel composition can vary depending on the batches of the components and the starting tumour material.

Then per well, and with 8 wells per agent or agent combination, a gel volume per well was added in an amount of from 12 to 15 microliters, with an addition of about 40 ul of medium on top.

Cell Culture Media

A standard cell culture medium was employed, comprising extra-cellular based hydrogel.

Preparation of 3D Screening Plates

Screenings were performed in 384 well plates, and filled by a liquid handling robot.

Drug Exposure

Drugs and drug combinations were added 24 hours day after plating of the tumour cell aggregates, at a volume to result in a total of 60 ul. Total drug exposure time ranged from 3 to 7 days. Next the tumour cell aggregates were fixed and stained for analysis.

Image Analysis

Images were captured by an inverted computer-controlled fluorescence microscope. Captured images were stored on a central data server, accessible by the OcellO Ominer™ 3D image analysis platform which allows direct parallel analysis of the 3D image stacks by its distributed computational design. This software analyses the structure of the objects (nuclei and cytoskeleton) detected in each well, and their relative positions.

Upon analysis, the output was checked to detect the quality of the raw images and the analysis method. The per-object measurements the software produced (such as its area) were subsequently aggregated per well and the data was coupled to the plate layout information (cell line, growth factor condition, treatment, etc.).

The above experiments showed that treatment with ipilimumab will only benefit a subset of patients, whereby this subset of patients show an in-vitro Selection Factor of below −30%.

1. A pharmaceutical composition for the treatment of patients having ovarian cancer, lung cancer or mesothelioma and showing a Selection Factor of −30% or below, comprising a therapeutically effective amount of ipilimumab, and optionally a pharmaceutically acceptable diluent or carrier, wherein the patient is selected on the basis of a positive response to an ex vivo three-dimensional (3D) patient derived tumour culture, the method comprising:
(a) preparing a three-dimensional, optionally size-normalised, tumour culture from a patient-derived tumour sample in a multitude of replicates;
(b) adding one or more immunotherapeutic agents to the culture, and
(c) culturing for a pre-defined time period; and
(d) determining the effect that the one or more immunotherapeutic agents has on the tumour cell aggregates by measuring the total area of objects in the culture that are above a threshold area associated with tumour cell aggregates, and the total area of objects that are below a threshold associated with immune cells, using three-dimensional imaging of the cell culture; wherein if following culturing with a composition comprising ipilimumab, the total area of the large objects decreases and/or the total area of the small objects increases relative to a control the patient is treated with ipilimumab.

2. A composition according to claim 1, wherein the patients have ovarian cancer or mesothelioma.

3. A composition according to claim 1, wherein the Selection Factor is determined according to the following steps:
(i) the sum of area of all tumour aggregates with an area of above about 420 μm² in each sample is calculated, and wherein it is determined if the sum of all areas is statistically significantly lower across the replicates comprising the same components;
(ii) the sum of area of all immune cells with an area smaller than about 160 μm² in each sample is calculated, and wherein it is determined if the sum of all areas is statistically significantly higher across the replicates comprising the same components, compared to the negative control; and
(iii) the effect on tumour aggregates is derived by calculating the percentage decrease of tumour aggregate area as a median of multitude of parallel tests within each replicate, and the median as calculated across the replicates, wherein the tumour aggregates are distinguished by an area threshold of 420 μm² and immune cells are distinguished by having their area smaller than 160 μm² according to formula I:
$$I ) Wilcoxon test : Does total area of large objects decrease ( p < 0.05 ) in treatment condition compared to the negative control ?$$
$$‐ No -> Selection Factor = 0$$
$$II ) Wilcoxon test : Does total area of small objects increase ( p < 0.05 ) in treatment condition compared to the negative control ?$$
$$‐ No -> Selection Factor = 0$$
$$If ( I ) \& ( II ) are met \to Selection Factor = 100 * \frac{\begin{matrix}median\_replicate ( {\sum }_{area > 422 μ m 2}^{Treatment} object area ) - \\ median\_replicate ( {\sum }_{area > 422 μ m 2}^{Negative ctl} object area )\end{matrix}}{median\_replicate ( {\sum }_{area > 422 μ m 2}^{Negative ctl} object area )}$$
wherein a Selection Factor below −30% indicates an effective treatment, and a patient responsive to the treatment

4. A composition according to claim 1, wherein the threshold associated with the tumour cell aggregates is above about 420 μm², and wherein the threshold associated with immune cells is about 160 μm².

5. The compositions according to claim 1, wherein the tissue sample may be directly employed after sampling and optional transport, or as a cryopreserved sample according to a standard protocol for preserving viability of human cells present in the sample, or wherein the sample is split into a fresh sample and a cryopreserved sample for correlation of the data at a later point in time.

6. The compositions according to claim 1, wherein step (d) comprises measuring the effect of the one or more immunotherapeutic agents on ex vivo patient derived 3D tumour cultures, by
i) staining of the cell culture with a fluorescence marker and measuring the fluorescence intensity to determine the total area of stained objects in the culture that are above about 420 μm² and below about 160 μm², and
ii) capturing a layered fluorescent image of the stained sample;
iii) and measuring the object intensity of the fluorescent surface areas in the sample;
and iv) determining the fluorescent surface areas.

7. The composition according to claim 6, wherein the sum of area of all tumour aggregates with an area of above about 420 μm² in each sample is calculated, and wherein it is determined if the sum of all areas is statistically significantly lower across the replicates comprising the same components.

8. The composition according to claim 7, wherein the sum of area of all immune cells with an area smaller than about 160 μm² in each sample is calculated, and wherein it is determined if the sum of all areas is statistically significantly higher across the replicates comprising the same components, compared to the negative control.

9. The composition according to claim 8, wherein the effect on tumour aggregates is derived by calculating the percentage decrease of tumour aggregate area as a median of multitude of parallel tests within each replicate, and the median as calculated across the replicates, wherein the tumour aggregates are distinguished by an area threshold of 420 μm² and immune cells are distinguished by having their area smaller than 160 μm² according to formula I:
$$I ) Wilcoxon test : Does total area of large objects decrease ( p < 0.05 ) in treatment condition compared to the negative control ?$$
$$‐ No -> Selection Factor = 0$$
$$II ) Wilcoxon test : Does total area of small objects increase ( p < 0.05 ) in treatment condition compared to the negative control ?$$
$$‐ No -> Selection Factor = 0$$
$$If ( I ) \& ( II ) are met \to Selection Factor = 100 * \frac{\begin{matrix}median\_replicate ( {\sum }_{area > 422 μ m 2}^{Treatment} object area ) - \\ median\_replicate ( {\sum }_{area > 422 μ m 2}^{Negative ctl} object area )\end{matrix}}{median\_replicate ( {\sum }_{area > 422 μ m 2}^{Negative ctl} object area )}$$
wherein a Selection Factor below −30% indicates an effective treatment, and a patient responsive to the treatment.

10. The composition according to claim 1, wherein step (d) further comprises segmenting the 3-dimensional culture into layers, capturing images of each layer, and deconvoluting the luminescence images of the layers to enhance the image contrast and create segmentation masks for individual cells and cell aggregates in the culture.

11. The composition according to claim 1, for use in a method for the treatment of female patients with recurrent epithelial ovarian cancer and showing a Selection Factor of −30% or below.

12. The composition according to claim 11, further comprising a synergistic and therapeutically effective amount of nivolumab and/or pembrolizumab and/or ADU-S100.

13. The composition according for the use according to claim 1, wherein ipilimumab is administered at a dose of about 0.3 mg/kg.

14. The composition according to claim 1, for use in treatment of a patient with metastatic or non-metastatic cancer, preferably lung cancer, peritoneal cancer, gastrointestinal cancer, pancreatic cancer, melanoma, glioblastoma, cervical cancer, ovarian cancer, liver cancer, bladder cancer, liver cancer, breast cancer, colon cancer, colorectal cancer, endometrial or uterine carcinoma, salivary gland carcinoma, kidney cancer, liver cancer, prostate cancer, vulvar cancer, thyroid cancer, mesothelioma, hepatic carcinoma and head and neck cancer, more preferably ovarian cancer, liver cancer or mesothelioma, more preferably ovarian cancer or mesothelioma, wherein the effect on tumour aggregates is derived by calculating the percentage decrease of tumour aggregate area as a median of multitude of parallel tests within each replicate, and the median as calculated across the replicates, wherein the tumour aggregates are distinguished by an area threshold of 420 μm² and immune cells are distinguished by having their area smaller than 160 μm² according to formula I:
$$I ) Wilcoxon test : Does total area of large objects decrease ( p < 0.05 ) in treatment condition compared to the negative control ?$$
$$‐ No -> Selection Factor = 0$$
$$II ) Wilcoxon test : Does total area of small objects increase ( p < 0.05 ) in treatment condition compared to the negative control ?$$
$$‐ No -> Selection Factor = 0$$
$$If ( I ) \& ( II ) are met \to Selection Factor = 100 * \frac{\begin{matrix}median\_replicate ( {\sum }_{area > 422 μ m 2}^{Treatment} object area ) - \\ median\_replicate ( {\sum }_{area > 422 μ m 2}^{Negative ctl} object area )\end{matrix}}{median\_replicate ( {\sum }_{area > 422 μ m 2}^{Negative ctl} object area )}$$
wherein a Selection Factor below −30% indicates an effective treatment, and a patient responsive to the treatment.

15. The composition for use according to claim 1, wherein the tumour cell culture comprises a naïve sample is derived from resected tumour specimen, tumour biopsies or malignant fluids, such as ascites or pleural effusion intravenous administration.

16. The composition for use according to claim 1, wherein the treatment further comprises a chemotherapeutic or immunotherapeutic molecule, a small molecule kinase inhibitor, a hormonal agent, a vaccine, ionizing radiation, ultraviolet radiation, cryoblation, thermal ablation, or radiofrequency ablation.
